(19) United States
(12) Patent Application Publication
(10) Pub. No.: US 2005/0055063 A1
(43) Pub. Date: Mar. 10, 2005

(54) Method and apparatus for the treatment of urinary tract dysfunction

(76) Inventors: Gerald E. Loeb, South Pasadena, CA (US); Hilton M. Kaplan, Beverly Hills, CA (US)

Correspondence Address: MCDERMOTT, WILL & EMERY;Suite 3400, 2049 Century Park East, Los Angeles, CA, 90067, US

(21) Appl. No.: 10/890,504

(22) Filed: Jul. 12, 2004

Related U.S. Application Data
(63) Continuation-in-part of application No. 10/200,273, filed on Jul. 22, 2002.
(60) Provisional application No. 60/486,573, filed on Jul. 11, 2003.
(60) Provisional application No. 60/306,992, filed on Jul. 20, 2001.
(60) Provisional application No. 60/307,725, filed on Jul. 25, 2001.

(51) Int. Cl. A61N 1/18

(52) U.S. Cl. 607/41

(57) ABSTRACT

The subject invention teaches the use of electrical stimulation of specific sensory nerves, such as the proximal urethral afferents to control urination. A wireless, injectable microstimulator may implanted into the soft tissues through which the sensory nerves pass. The sensory nerves supplying the proximal urethra are stimulated by a microstimulator implanted adjacent to the prostatic urethra within the substance of the prostate gland in males, and distal to the bladder neck in females. The activity induced in these nerves causes the spinal cord to generate reflex responses that result in contractions of the detrusor muscle and relaxation of the sphincter, emptying the bladder. The invention also includes methods of implanting and/or testing microstimulators at a target location. The invention also includes the use of sensory devices to effect the microstimulators or alert the user as to the status of the bladder.

### CROSS-REFERENCE To RELATED APPLICATIONS

This application claims priority to U.S. Provisional Patent Application Ser. No. 60/486,573, filed Jul. 11, 2003, entitled “Treatment of Urinary Tract Dysfunction.” This application is also a continuation-in-part of U.S. patent application Ser. No. 10/200,273 filed Jul. 22, 2002 entitled, “Method and Apparatus for the Treatment of Urinary Tract Dysfunction.” This application is also related to two prior U.S. Provisional Applications Ser. No. 60/306,992, filed Jul. 20, 2001, and Ser. No. 60/307,725, filed Jul. 25, 2001, entitled “Method and Apparatus for the Treatment of Urinary Tract Dysfunction. All of these applications are incorporated herein by reference.

### BACKGROUND OF THE INVENTION

1. Field of the Invention

The invention relates to methods and associated apparatus which are useful for the treatment of urinary tract dysfunction. More particularly, the invention is directed to the use of an apparatus to control the filling and/or emptying of the bladder. More particularly, the invention is directed to the stimulation of the proximal urethral afferents for initiating and maintaining the micturition reflex. Additionally, the invention is directed to methods for the insertion and testing of microstimulators in the proximity of the proximal urethral afferents. Finally, the invention is directed to devices and methods for sensing the bladder distention so that filling and/or emptying of the bladder may be monitored.

2. Background and State of the Art

Various dysfunctions of the urinary tract and its associated muscles and nerves result in the common clinical problem of urinary incontinence. Such dysfunctions may arise spontaneously in otherwise healthy individuals, but they are particularly common after various forms of damage to the spinal cord or peripheral nerves. The resulting incontinence interferes with the social life and health care of the patient. The neural mechanisms responsible for these dysfunctions are not fully understood, but recent advances in understanding the neurophysiology of the intact system has provided important clues as to how these dysfunctions might be treated.

The bladder acts as a storage reservoir for urine generated by the kidneys. The bladder walls contain a muscle called the detrusor, which contracts to generate pressure and expel urine. The bladder wall also contains stretch receptors, which send signals about the distension of the bladder to the spinal cord. The interval of time between episodes of urination depends on the available volume of the reservoir. In normal adults, the capacity of the bladder is at least 500-700 cc. As the bladder starts to approach this capacity, the spinal cord reacts to the signals from the stretch receptors by activating the detrusor muscle. A person with an intact nervous system will be aware of both the distension and the pressure produced by the muscle contractions. If it is inconvenient to urinate, the person can voluntarily contract the external urethral sphincter muscles to prevent urination until it is convenient to do so.

Patients with damage to the spinal cord and other central pathways suffer from various dysfunctions of the processes described above. Often these patients are unable to sense or respond voluntarily to the contractions of a full bladder. When bladder contractions do occur, they are accompanied by an excessive reflexive response of the internal and/or external sphincter muscles, preventing urination even when desired. Still other patients fail to produce active bladder contractions even when the bladder is full; urine leaks out by overflow and the bladder never empties fully. Many patients suffer from a combination of these dysfunctions.

Many strategies have been proposed to use electrical stimulation to alter and correct dysfunction of the neuromuscular components of the urinary tract, some of which are now used in clinical practice. Conventional technologies for stimulating nerves include transcutaneous magnetic fields (induction of eddy currents in tissues by intense, pulsed magnetic fields created in externally affixed induction coils), transcutaneous electrical currents (applied via electrodes affixed to the skin or inserted into the vagina or rectum), percutaneous electrical currents (via wires injected through the skin that can be connected to external electronic stimulators) and fully implanted stimulators (pacemaker-like devices with leads routed subcutaneously to stimulating electrodes surgically affixed to the target structure). The Vocare® by Finetech Ltd. (England) is a surgically implanted device available in Europe that stimulates various of the sacral roots of the spinal cord. This tends to produce both detrusor and sphincter contraction, but emptying of the bladder can be accomplished in a series of contractions because the sphincter relaxes before the detrusor does, allowing a spurt of urine to escape.

Because the nerves that control bladder function are located deep in the pelvis, transcutaneous magnetic and transcutaneous electrical stimulation are often unacceptable because of the many other excitable nerves located superficial and adjacent to the target nerves. Stimulation of these adjacent nerves may lead to undesirable sensory perceptions or unwanted motor effects. Percutaneous wires are usually unacceptable for chronic use, particularly in the perineal region of the body where they are subject to infection and mechanical damage. Research to date has focused on surgical implantation of stimulating electrodes in, on or near main nerve trunks such as the sacral roots, pudendal nerve or the spinal cord itself. This requires the surgical routing of electrical leads from the electrodes to implanted electrical stimulators similar to cardiac pacemakers. Such surgical intervention is often feasible only for relatively large nerves that happen to run in places where they can be approached without endangering adjacent delicate or vital structures. Sites suitable for such intervention include the pudendal nerve as it passes the ischium, the spinal roots as they pass through the sacral foramena, and the spinal cord within the dural sheath. One disadvantage of all of these sites is that they contain a mixture of neurons subserving various sensory and motor functions. This often makes it difficult to achieve the desired effects without producing undesirable side effects from inadvertent stimulation of inappropriate neurons. For example, the implantation of the Vocare system usually requires cutting of the dorsal sacral roots, which eliminates sexual function. Another common disadvantage is that they generally require surgical intervention to implant the required devices, which entails high costs and risks of patient discomfort, infection and morbidity.

Accordingly, the need remains for devices and methods for the placement of microstimulators to induce, maintain and control micturition.

### SUMMARY OF THE INVENTION

As disclosed above, urination is permitted to occur by voluntary relaxation of the external urethral sphincter. When urination is permitted to occur, the flow of urine through the urethra is detected by other sensory receptors in the urethral walls. Activity in these urethral afferent neurons gives rise to reflex responses in the spinal cord that further excite the detrusor (bladder wall) to contract and to relax both the internal and external urethral sphincters thus promoting the complete emptying of the bladder. It has recently been demonstrated that the proximal urethral afferents, rather than the distal ones, are predominantly involved in this pathway by the activation of spinal neural circuits. Gustafson, et al., “Development of a Non-Invasive, Catheter Based Method to Activate Urethral Afferents”, Proceedings of the 7th Annual IFESS Conference, Ljubljana, Slovenia, Jun. 25-29, 2002; Gustafson, et al., “Generation of Bladder Contractions via Electrical Stimulation of Urethral Afferent Nerves and Intra-Urethral Stimulation”, Proceedings of the 2nd Joint EMBS/BMES Conference, Houston, Tex., USA, Oct. 23-26, 2002; Gustafson et al., “A Catheter Based Method to Activate Urethral Sensory Nerve Fibers”, Journal of Urology. 170(1):126-9, July 2003. In male patients these proximate urethral afferents are typically located within the prostate gland, and more specifically, in the prostate proximate to the prostatic urethra, and in females in the proximal several centimeters of the urethra, typically proximate to the internal urethral sphincter, such as near the bladder neck.

A new class of implantable medical devices, such as the BION™ microstimulator, makes it possible to create accurately localized and precisely graded electrical fields within virtually any body structure. Each microstimulator may include electrical stimulation circuitry and electrodes configured in a form that is suitable for injection, such as through a hypodermic needle. There need not be any attached leads to receive power or commands or to route stimulation pulses to distant electrodes. Microstimulators may receive power by inductive coupling to an externally applied radio frequency (“RF”) magnetic field. They may receive digital command signals by detecting and decoding modulations of an RF carrier. The electronic circuitry in the microstimulator may use the power and data immediately to generate the required electrical stimulation currents in the adjacent tissue by passing current through the integral electrodes, or it may store power and data by various conventional means to enable the generation of output pulses when the RF field is not present. The packaging and materials of the microstimulator may be selected and designed to protect its electronic circuitry from the body fluids and to avoid damage to the electrodes and the surrounding tissues from the presence and operation of the microstimulator in those tissues. Thus, microstimulators, such as BIONs may be well suited to delivering well-controlled and stable electrical stimulation to nerves in sites that are not amenable to stimulation by conventional technologies as described above.

The invention may include the use of one or more microstimulators implanted into soft tissues of the pelvis to effect spinal reflex mechanisms that modulate the state of the muscles that control pressure and flow in the bladder and/or urethra. In one embodiment, a microstimulator may be located in the vicinity of the proximal urethra where it can excite sensory fibers, such as proximal urethral afferents, whose reflex actions tend to initiate or promote contraction of the detrusor and relaxation of the internal and/or external urethral sphincters. This microstimulator may be positioned within the prostate gland in males; and proximate to the internal urethral sphincter in females.

In another embodiment, a second microstimulator may be located proximate to the dorsal penile nerve in a male or clitoral nerve in a female, whose activation tends to elicit reflexes that inhibit bladder contractions.

In another embodiment, a sensor may be placed proximate to the bladder to detect bladder distention.

An apparatus for exciting proximal urethral afferents to induce micturition may include a first injectable microstimulator comprising a chamber separating a plurality of exposed electrodes for delivering controllable electrical current in the area of the proximal urethral afferents; and an electronic circuit within said chamber in communication with said electrodes for generating the controllable electrical current in response to a control signal. A receiving antenna may be within said chamber in communication with said electronic circuit for receiving a control signal. The apparatus may include a controller for generating the control signals and a transmitting antenna in communication with said controller for transmitting a control signal to said receiving antenna, wherein the control signal includes instructions to generate an electrical pulse having a frequency in the range of 2 to 20 pulses per second until the bladder is emptied.

The injectable microstimulator may be adapted for implantation proximate to or within the prostate of a male patient.

The injectable microstimulator may be adapted for implantation proximate to the internal urethral sphincter of a female patient.

A second injectable microstimulator may be included that is injectable in an area in a human body which inhibits micturition. A transmitting antenna may be in communication with said controller for transmitting a control signal to said receiving antenna, wherein the control signal includes instructions to generate an electrical pulses having a frequency of about 20 pulses per second for about 5 seconds followed by a rest of about 5 seconds until bladder emptying is desired.

The second injectable microstimulator may be adapted for stimulation of the dorsal penile nerve. The second injectable microstimulator is adapted for stimulation of the clitoral nerve.

A second injectable microstimulator may be included wherein said first injectable microstimulator is adapted for implantation in the prostate and said second injectable microstimulator is adapted for implantation proximate to the dorsal penile nerve.

A second injectable microstimulator may be included wherein said first injectable microstimulator is adapted for implantation proximate to the internal urethral sphincter and said second injectable microstimulator is adapted for implantation proximate to the clitoral nerve.

The transmitting antenna may be a coil of a circumference suitable for positioning around the base of the penis for controlling said first injectable microstimulator and a second injectable microstimulator.

A user interface may include a user-activated control switch for initiating stimulation of the proximal urethral afferents in order to induce or maintain micturition.

An apparatus for exciting proximal urethral afferents to induce micturition comprising an injectable microstimulator having a plurality of exposed electrodes for delivering controllable electrical current having a frequency in the range 2 to 20 pulses per second until the bladder is emptied in the area of the proximal urethral afferents which cause the urethral afferents to induce micturition by inducing detrusor contraction or urethral sphincter relaxation.

An apparatus for exciting proximal urethral afferents to induce micturition may include a first microstimulator implanted in proximity to proximal urethral afferents having a chamber separating a plurality of exposed electrodes for delivering controllable electrical current in the area of the proximal urethral afferents in response to at least one control signal and a receiving antenna within said chamber for receiving at least one control signals; a control unit for generating at least one control signal; and a transmitting antenna in communication with said control unit for transmitting the at least one control signals to said receiving antenna, wherein the control signal includes instructions to generate an electrical pulse having a frequency in the range of 2 to 20 pulses per second until the bladder is emptied.

The first microstimulator may be configured for implantation proximate to or within the prostate, and a second microstimulator may be configured for implantation proximate to the dorsal penile nerve.

The first microstimulator may be configured for implantation proximate to the internal urethral sphincter, and a second microstimulator may be configured for implantation proximate to the clitoral nerve.

The transmitting antenna may be a coil, wherein said coil is of a circumference suitable for positioning around the base of the penis and adapted for controlling said first microstimulator and a second microstimulator.

A user interface may be included, including a user-activated control switch for initiating stimulation of the proximal urethral afferents in order to induce or maintain micturition.

A method for inducing micturition may include positioning a leadless microstimulator proximate the proximal urethra; and electrically stimulating a proximal urethral afferent to induce at least one of contraction of the detrusor, relaxation of the internal urethral sphincter or relaxation of the external urethral sphincter.

A method for exciting proximal urethral afferents to induce micturition may include generating an electrical signal by a control unit; delivering the signal to a transmitting antenna; generating an electrical signal in the first microstimulator in the area of urethral afferents having a frequency in the range of 2 to 20 pulses per second until the bladder is emptied; and inducing micturition.

An electrical signal may be delivered to a proximal urethral afferent through a plurality of microstimulators.

Micturition may be inhibited with a second microstimulator that receives a signal from the control unit and generates an electrical signal for inhibiting micturition.

Stimulation of the urethral afferents may be included in order to induce or maintain micturition.

A first microstimulator may be implanted in the prostate in a male patient.

A first microstimulator may be implanted proximate to the internal urethral sphincter in a female patient.

A first microstimulator may be injected in the area of the proximal urethral afferents.

A second microstimulator may be injected in the area of the dorsal penile nerve to inhibit micturition.

A second microstimulator may be injected into a human recipient in the area of the clitoral nerve to inhibit micturition.

The injecting may be through an injection device.

A method of implanting a microstimulator proximate to a proximal urethral afferent nerve in a male patient may include inserting an injection device having a cannula through the perineum; positioning the microstimulator in the injection device cannula proximate to an urethral afferent in the prostate; and releasing the microstimulator from the injection device cannula proximate to the urethral afferent in the prostate.

Prior to releasing the microstimulator from the injection device cannula proximate to the urethral afferent in the prostate, the microstimulator may be tested for excitation of the urethral afferents by observing at least one of the contraction of the detrusor or relaxation of the urethral sphincter.

A method of implanting a microstimulator proximate to a proximal urethral afferent nerve in a female patient may comprise inserting an injection device having a cannula through the vagina; positioning the microstimulator in the injection device cannula proximate to an urethral afferent proximate to the internal urethral sphincter; and releasing the microstimulator from the injection device cannula proximate to the urethral afferent proximate to the internal urethral sphincter.

Prior to releasing the microstimulator from the injection device cannula proximate to the urethral afferent, the microstimulator may be tested proximal to the internal urethral sphincter for excitation of the urethral afferents by observing at least one of the contraction of the detrusor or relaxation of the internal or external urethral sphincter.

A method for positioning a microstimulator proximate to the proximal urethral afferents to induce micturition may include inserting a distal tip of a cannula having the microstimulator retained within a cannula lumen through the perinueum until the implant reaches a testing position proximate to the proximal urethral afferents; testing the microstimulator while within the cannula lumen at the testing position to determine whether the implant is functioning effectively to induce micturition; and discharging the microstimulator from the lumen of the cannula at the testing location if the testing reveals that the implant is functioning effectively at to induce micturition.

The microstimulator may be moved within the cannula lumen to a new position if testing shows the implant is not located at effective position and re-testing the implant while within the cannula lumen at the testing position to determine whether the implant is functioning effectively to induce micturition.

The movement of the microstimulator may include longitudinal movement relative to the target location.

The movement of the microstimulator may include axial rotation relative to the target location.

The microstimulator may be maintained at the testing location during the discharge of the implant from the lumen.

A method for measuring bladder distention and controlling micturition may include implanting a first microstimulator proximate to the bladder wall; implanting a second microstimulator proximate to a proximal urethral afferent; emitting a electrical pulse from one of the first or second microstimulator; detecting the strength of the electrical field at the other of the first or second microstimulator; calculating the distension of the bladder from the strength of the electrical field detected to determine the presence of urine in the bladder; and emitting an electrical pulse from the second microstimulator to excite the proximal urethral afferent to induce micturition if urine is present in the bladder.

The first microstimulator may be implanted proximate to the dome of the bladder.

Information reflecting the strength of the electrical field detected may be conveyed to an external controller for calculating the distention of the bladder.

The calculation of the distention of the bladder may be transmitted to a user interface.

An electrical pulse may be emitted at a third microstimulator implanted proximate to a dorsal penile nerve to inhibit micturition.

An electrical pulse may be emitted at a third microstimulator implanted proximate to a clitoral nerve to inhibit micturition.

### BRIEF DESCRIPTION OF THE DRAWINGS

FIGS. 1A and B are schematic diagrams displaying the components of the urinary tract and a combination of positions of microstimulators/sensors in one embodiment of the invention in a male (A) and female patient (B), respectively.

FIGS. 2A and B are schematic diagrams displaying one method for implanting a microstimulator/sensor according to one embodiment of the invention in a male (A) and female patient (B), respectively.

FIGS. 3A-C are of embodiments of an injection device for positioning a microstimulator proximate to a proximate urethral afferent. FIG. 3A is a longitudinal cross-section of the distal end of the injection device having a microstimulator loaded in the cannula lumen; FIG. 3B is a longitudinal view of the distal end of an injection device; FIG. 3C is a longitudinal view of the distal end of an injection device.

FIG. 4A is a longitudinal view of one embodiment of an injection device; FIG. 4B is a cross-sectional view of the distal end of the injection device having a detent; FIG. 4C is a longitudinal view of one embodiment of a microstimulator; FIG. 4D is a front view of one embodiment of a microstimulator.

FIG. 5A is a longitudinal view and cross-section of the distal end of one embodiment of an injection device having an implant loaded in the lumen; FIG. 5B is a longitudinal view of one embodiment of a probe for use in an injection device; FIG. 5C is an inset of a probe distal end tab configuration; FIG. 5D is a side view of one embodiment of an implant; FIGS. 5E and 5F are cross-sectional views of probe/implant configurations.

### DETAILED DESCRIPTION OF ILLUSTRATIVE EMBODIMENTS

While the specification describes particular embodiments of the present invention, those of ordinary skill can devise variations of the present invention without departing from the inventive concept.

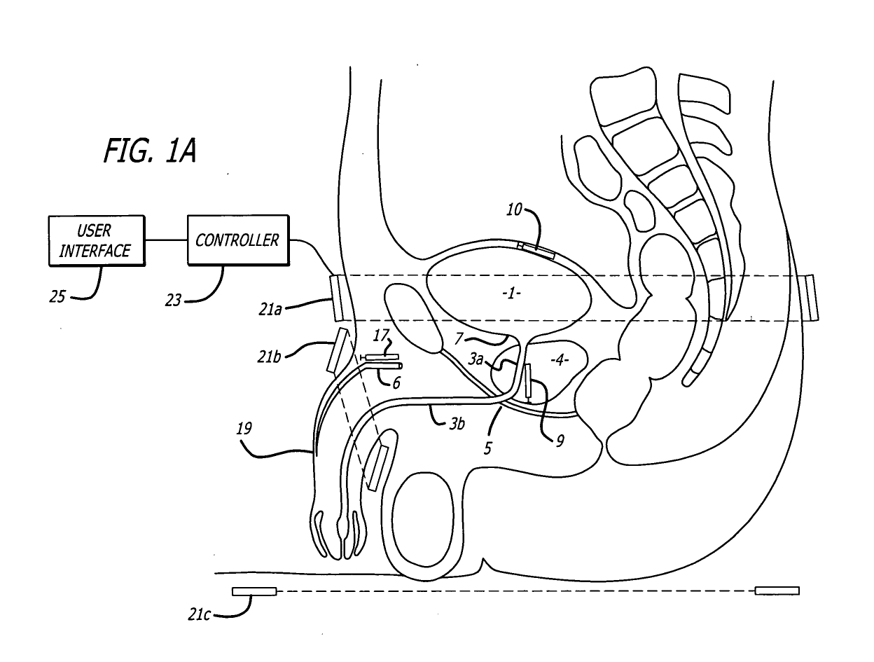

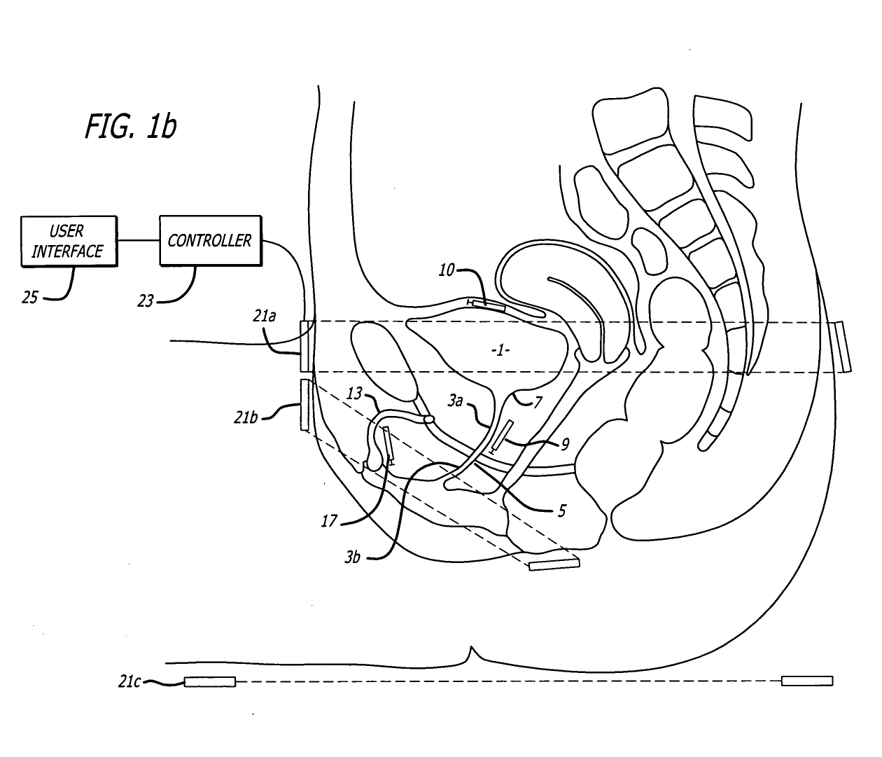

FIGS. 1A and B are schematic diagrams displaying the components of the urinary tract and a combination of positions of microstimulators/sensors in one embodiment of the invention in a male (A) and female patient (B), respectively. Referring to FIGS. 1A and 1B, the main anatomical components of the lower urinary tract include the bladder (1), the urethra (3) and the external urethral sphincter (5), which is comprised of the musculature of the pelvic floor and surrounds the urethra (3) as it passes through this musculature. The portion of the urethra (3) that is proximal to the external urethral sphincter (5) is the proximal urethra (3a) and the portion that is distal to the external urethral sphincter (5) is the distal urethra (3b). Near the exit of the bladder (1) into the urethra (3) there is an internal urethral sphincter (7) which is integral to the bladder neck. These structures are innervated by several different nerves subserving a wide range of sensory and motor functions that are interconnected in the spinal cord by many different spinal circuits. Only the most pertinent components are described herein and the details of their functions have been greatly simplified.

For voiding. In one embodiment of the invention, it is contemplated to induce or maintain micturition by stimulation of the proximal urethral afferents, via at least one microstimulator implanted adjacent to the proximal urethra (3a). It should be noted that the distribution of the proximal urethral afferent nerves is as a plexus, and may be variable between patients. Any one microstimulator may be anatomically positioned so as to be in the proximity of any one or several proximal urethral afferents. Further, any one microstimulator may be functionally tested to ensure it functionally effects any one or several proximal urethral afferents.

In one embodiment of the invention, a microstimulator (9), such as a BION or the like may be used to stimulate a proximal urethral afferent. This embodiment may permit intermittent inductive powering at the time of urination, without requiring an external control source (i.e., coil (21)) to be worn at other times. Intermittent stimulation of a proximal urethral afferent alone may be suitable to induce micturition for males and females, so as to minimize undesirable sphincter contractions.

Proximal urethral afferents within the prostate gland (male)(4) or just distal to the internal urethral sphincter (females) (7) convey sensory information from the proximal urethra (3a) to the spinal circuits whose reflex outputs tend to excite contraction of the detrusor muscle of the bladder (1) and reduce activity in the external urethral sphincter (5) and internal urethral sphincter (7). Once they leave the vicinity of the proximal urethra (3a), the anatomical course of the urethral afferents is not known and may not be surgically accessible in isolation. However, in one embodiment of the present invention, electrical stimulation may be applied in the vicinity of the proximal urethra (3a) itself by at least one microstimulator (9) located or implanted immediately adjacent to the proximal urethra (3a) within the prostate gland (male)(4) or just distal to the internal urethral sphincter (7) in females to induce contraction of the detrusor (bladder) (1) and/or relaxation of the internal urethral sphincter (7) and or the external urethral sphincter (5) to permit mictrition.

For preventing incontinence. In one embodiment, the implantation of two devices may also allow the ability to switch between two sites of stimulation that exert, respectively, excitatory (proximal urethral (3a) afferent nerves) and inhibitory (dorsal penile (6)/clitoral nerves (13)) reflex effects on bladder contraction. Many patients have a mix of dysfunctions that may change over time in response to progression of their underlying neurological problems and plastic changes in the genitourinary tract that may occur as a result of chronic use of the treatment disclosed herein.

Still referring to FIGS. 1A and 1B, in one embodiment a microstimulator (17) is configured to inhibit bladder detrusor contractions in order to facilitate its gradual filling with urine produced by the kidneys. This may be accomplished by applying continuous or intermittent electrical stimulation in a regular pattern to the dorsal penile nerves (6) in the male or dorsal clitoral nerves (13) in the female via microstimulator (17) implanted adjacent to these nerves. In the male, the external surface of the penis is innervated by the two dorsal penile nerves (6) that run in parallel along the dorsal surface of the penis (19). One or both penile nerves (6) may be electrically stimulated by a microstimulator (17) implanted between or adjacent to the dorsal penile nerves (6), which results in an inhibition of spinal circuits and a consequent prevention or reduction of bladder detrusor contraction.

In males, the dorsal penile nerve (6) may be excited by at least one microstimulator positioned near the base of the penis (19) and aligned axially with the long axis of the penis, permitting the implant to be powered and controlled by a small circumferential coil (21b) suitable for placement around the base of the penis (19) for example. In one embodiment, this coil (21a & c) may be of a size to allow penetration of the magnetic field required to power also microstimulator (9) which is more deeply implanted in the perineum, for example in the prostate (4).

In the female, the clitoral nerve (13) is functionally analogous to the dorsal penile nerve (6) in the male. The clitoral nerve (13) may be electrically stimulated by a second microstimulator (17) implanted proximate to the clitoral nerve (13), which results in an inhibition of spinal circuits and a consequent prevention or reduction of bladder detrusor contraction.

When the patient is ready to urinate, user interface (25) may permit the patient to change the state of controller (23) so that a pattern of stimulation of the proximal urethral afferents by microstimulator (9) begins and/or stimulation of the dorsal penile nerves (6)/clitoral nerve (13) by microstimulator (17) is discontinued. If the coil configuration (21b in FIG. 1A) around the base of the penis is being used to maintain continence by inhibiting bladder contraction, then when micturition is desired, coil (21b) can be repositioned to the base of the perineum so as to be able to send commands effectively to microstimulator (9) within the prostate (4).

Stimulation pattern. One example of a stimulation pattern for use with microstimulator (9) may be a train of pulses at 2 to 20 pps which may continue until the bladder is empty and the flow of urine ceases. The amplitude of the stimulation pulses may be set initially by the prescribing therapist in a urodynamic examination by determining the level that results in reflexive contraction of the bladder, as determined by measuring increases in bladder pressure when the bladder is full, or relaxation of the external urethral sphincter (5) as measured by an instrumented catheter such as is commonly used in the clinical measurement of urodynamics.

One example of a stimulation pattern for use with microstimulator (17) may be a train of pulses at 20 pps for 5 sec. followed by a 5 sec. pause. The pulse train and pause length may be selected to prevent the spinal circuits from habituating to the stimulation, but is not so long as to allow the reflex inhibition of the bladder to wear off. The amplitude of the stimulation pulses may be selected such that the microstimulator has the desired function effect and may be set initially by the prescribing therapist by observing the reflexive contraction of the pelvic floor muscles that tends to be elicited by activation of the dorsal penile nerves (6) or clitoral nerve (13) in males and females respectively. Direct measurement of bladder contraction and relaxation can be obtained via an instrumented catheter such as is commonly used in the clinical measurement of urodynamics.

Microstimulator operation. Microstimulators (9) and (17) may receive power and command signals by inductive coupling of a modulated alternating magnetic field created by an external source, such as a coil (21). The electrical signals required to generate this magnetic field may be produced by a controller (23), whose state may depend on inputs from the patient received via user interface (25) and/or may depend on signals received from other sensory microstimulators. Various specific methods and electronic circuits required to achieve the required functionality of the external and implanted elements (9, 10, 17, 21, 23, 25) are well-known and well-described in the prior art, examples of which include, but are not limited to those disclosed in Loeb, et al., “BION™ System for Distributed Neural Prosthetic Interfaces,” Medical Engineering and Physics 23: 9-18 (2001); Younghee Lee et al., “Detrusor and Blood Pressure Responses on Dorsal Penile Nerve Stimulation During Hyper-Reflexic Contraction of Bladder in Patients with Cervical Cord Injury,” Proceedings of the 6th Annual Conference of the International Functional Electrical Stimulation Society: Oral Session II: Neural Prostheses II: Sensory & Organ Systems; Gustafson, et al., “Bladder Contractions Evoked by Electrical Stimulation of Pudendal Afferents in the Cat,” Proceedings of the 6th Annual Conference of the International Functional Electrical Stimulation Society: Oral Session II: Neural Prostheses II: Sensory & Organ Systems; Chapin et al., “Neural Prostheses for Restoration of Sensory and Motor Function,” Loeb et al., BION™ “Implants for Therapeutic and Functional Electrical Stimulation,” U.S. Pat. Nos. 5,193,540, 5,193,539, 5,312,439, 5,324,316, 5,405,367, 5,571,148, 5,697,076, 6,051,017, 6,061,596, PCT Publications WO/98/37926, 98/43700, 98/43701 herein incorporated by reference.

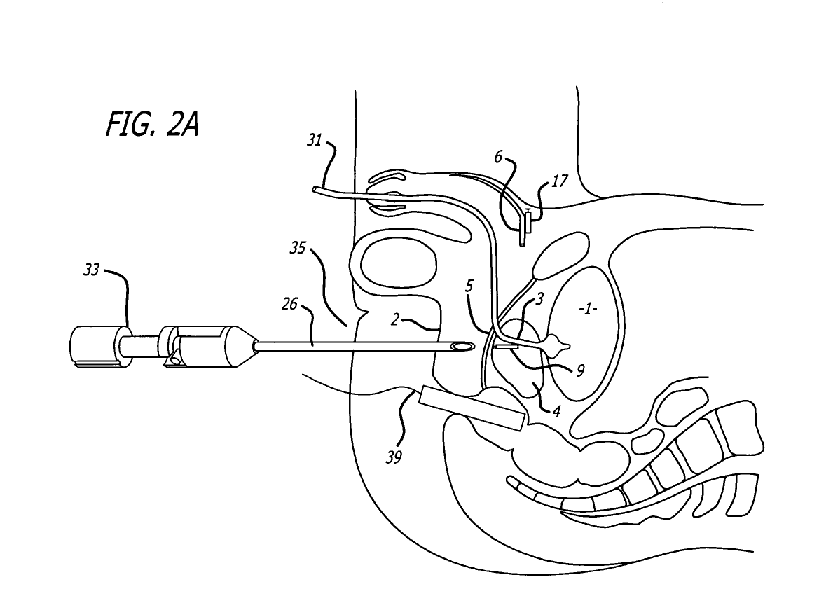

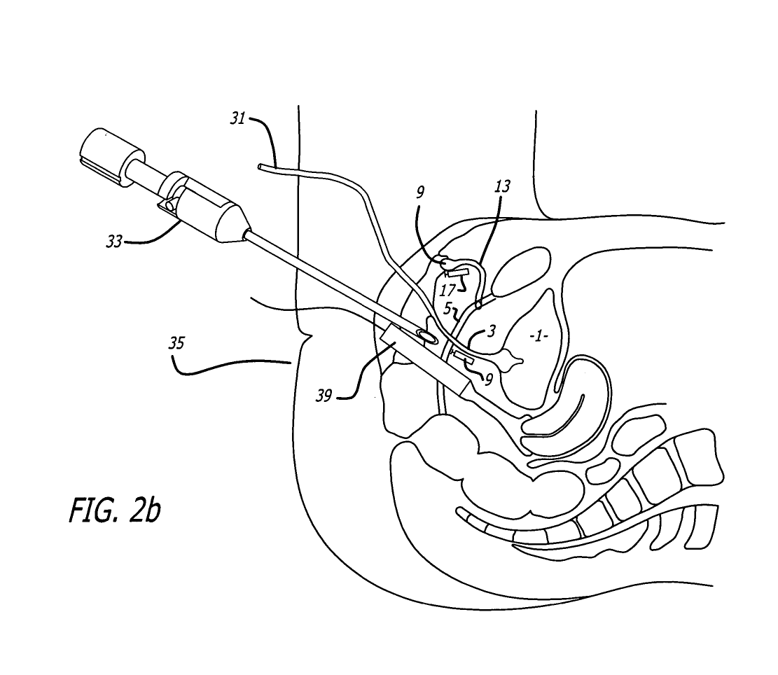

In one embodiment, the controller (23) may include a storage component whereby one or more programs of stimulation pulses that have been devised by a physician can be retained electronically therein and generated as required by the patient. In one embodiment of the invention, microstimulators may have a storage component for power and control signals so that they can generate stimulation pulses even when the controller (23) is not physically present. In that embodiment a coil (21) and a controller (23) may be used intermittently to provide power to recharge a power storage component such as rechargeable lithium ion cells and to transmit data regarding the required stimulation parameters to a microstimulator (9/17). FIGS. 1 and 2 illustrate exemplary alternative placements for coil (21), including around the hips or waist (21a), around the penis or vulva (21b) and within a seating appliance such as a wheelchair or toilet seat (21c). Such interfaces are taught also in U.S. Pat. No. 6,061,596, which is incorporated herein by reference.

Method of implantation. FIGS. 2A and B are schematic diagrams displaying one method for implanting microstimulator (9) according to one embodiment of the invention in a male (A) and female patient (B) respectively.

In particular for the proximal urethral afferents in the male, one or more microstimulators may be implanted alongside the prostatic urethra (3a), in the substance of the prostate gland (4), through which the sensory nerve fibers run.

In one embodiment, the microstimulator may be implanted into or proximate to the body of the prostate gland (4) by an injection device, which may include a cannula having a lumen for housing the microstimulator. For example, a standard urological approach used for implanting radiotherapeutic beads transperinealy may be used (such as described in Vicini, et al, “A Comprehensive Review of Prostate Cancer Brachytherapy: Defining an Optimal Technique”, Int. J. Radiation Oncology Biol. Phys., Vol. 44, No. 3, pp. 483-491, 1999, herein incorporated by reference).

Also, an implant, such as the microstimulator (9/17) or sensor (10) may be implanted or injected using a hypodermic needle such that the implant may be fitted within the cannula and pushed from the distal end of the needle to the target location.

Also, a delivery cannula may be used such that the implant may be fitted within the cannula and pushed from the distal end of the cannula, such as by a trochar to release the implant at the target location. In one embodiment, implant or sensor positioning may be achieved by first inserting a trochar surrounded by an outer plastic sheath into the body. A conductive distal tip of the trochar may be used to electrically stimulate a test location to evoke a response. The trochar/outer sheath assembly may be moved and electrical stimulation may be repeated until the desired response is achieved. The trochar may then be removed from the outer plastic sheath while holding the sheath in position in the body. An implant or sensor may then be manually inserted into the outer sheath and pushed out past the outer sheath distal end with an inner blunt push rod. The outer sheath and push rod may then be removed from the patient leaving the implant behind, such as proximate to a proximal urethral afferent.

In another embodiment, one end of an elongated cylindrical implant may be wedged into the end of a plastic inner sheath. When the trochar is removed from the outer sheath, the assembly consisting of the implant and inner sheath may be inserted in its place, leaving the implant protruding from the end of the outer sheath but still captured in the end of the inner sheath. In this position, it may be possible to activate the implant for testing purposes and to make small adjustments in position, such as decreasing depth. If the location is judged acceptable, the implant may be extruded from the end of the inner sheath by a blunt push rod located within the inner sheath and the entire injection device (outer sheath, inner sheath and push rod) may be removed from the body. If the location is not acceptable, the assembly consisting of the implant and inner sheath may be removed from the outer sheath and replaced by the sharp trochar before any significant repositioning of the injection device can be attempted.

Also, this implantation approach may use an injection device, such as the “BION Insertion Tool” (33) the structure of which and methods of use may be as described in a separate U.S. patent applications Ser. No. 10/461,560 filed Jun. 12, 2003 entitled, “Injection Devices and Methods for Testing Implants Prior to Positioning”; and Ser. No. 10/461,132 filed Jun. 12, 2003, entitled “Injection Devices for Unimpeded Target Location Testing,” herein incorporated by reference.

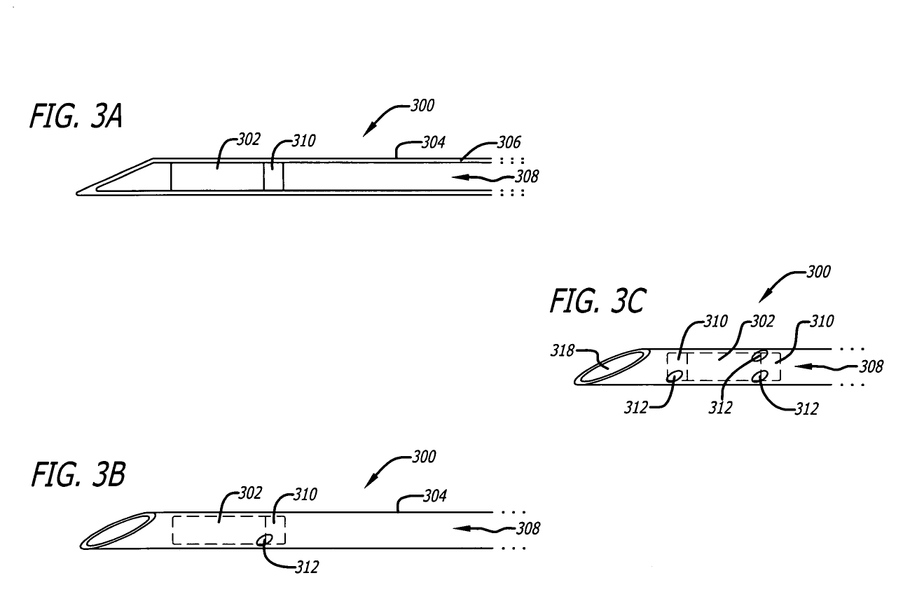

In one embodiment, it may be desirable to position the implant or sensor used in the present invention with a high degree of longitudinal or rotational accuracy. FIGS. 3A-C are of embodiments of an injection device (300) for positioning a implant (302), such as microstimulator (9) proximate to the proximal urethral afferents. The injection device (300) may include a cannula (304) having a substantially cylindrical cannula wall (306) forming a cannula lumen (308). An implant may be configured for positioning within the cannula lumen (308) and the implant may have at least one external electrode (310) (FIG. 3A). Further, at least one fluid communication channel (312) (“channel”) may be formed in the cannula wall (306) to permit interstitial fluid from the target location to enter into the cannula lumen (308) and contact the implant (302) (FIG. 3B). A channel (312) may be formed at a location along the cannula length, such that the channel (312) is substantially aligned with the external electrode (310).

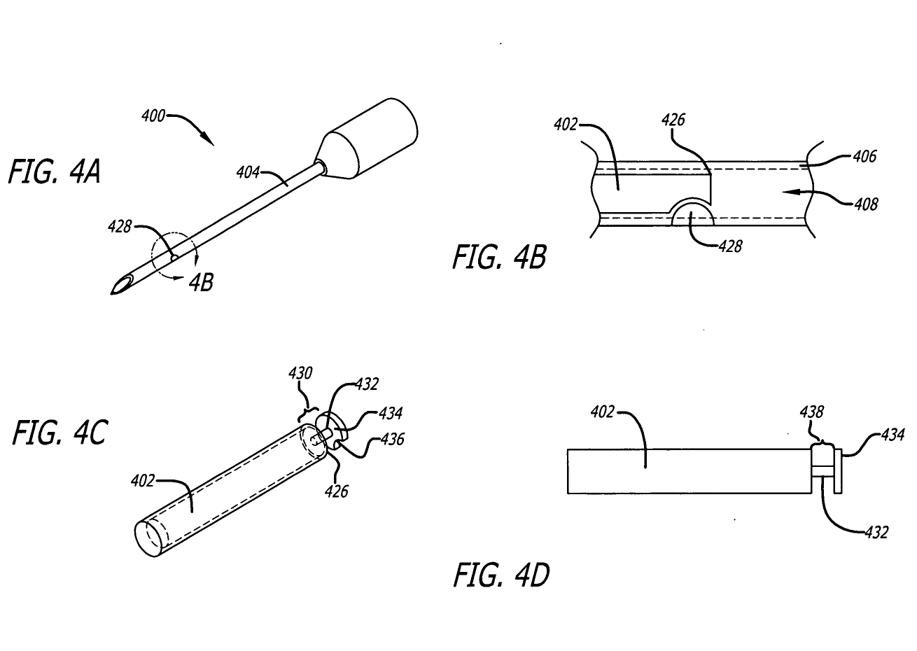

As shown in FIG. 4A, in one embodiment, the injection device (400) may include a cannula (404) and an implant (402) positioned within the cannula lumen (408), such that an implant end surface (426) is configured to releasably engage a surface within the cannula lumen (408). As depicted in FIG. 4B, in one embodiment, the cannula lumen (408) may be modified to include a detent (428). Further, the implant surface (426) may be modified to form a retaining member (430) (FIGS. 4C & D). The retaining member (430) may be integral to the implant (402) or may be formed as a separate structure which is then attached to the implant surface (426). In one embodiment, the retaining member (430) may include a post (432) and an annular ring (434), having a notch (436) therein (FIG. 4C). The post (432) length may be selected such that the detent (428) fits within a detent space (438) formed between the implant surface (426) and the annular ring (434). The notch (436) in the annular ring (434) may be formed in any shape having notch cross-section that is compatible with the detent cross section, such that the notch (436) can move slidably past the detent (428) when the detent (438) and notch (436) are axially aligned.

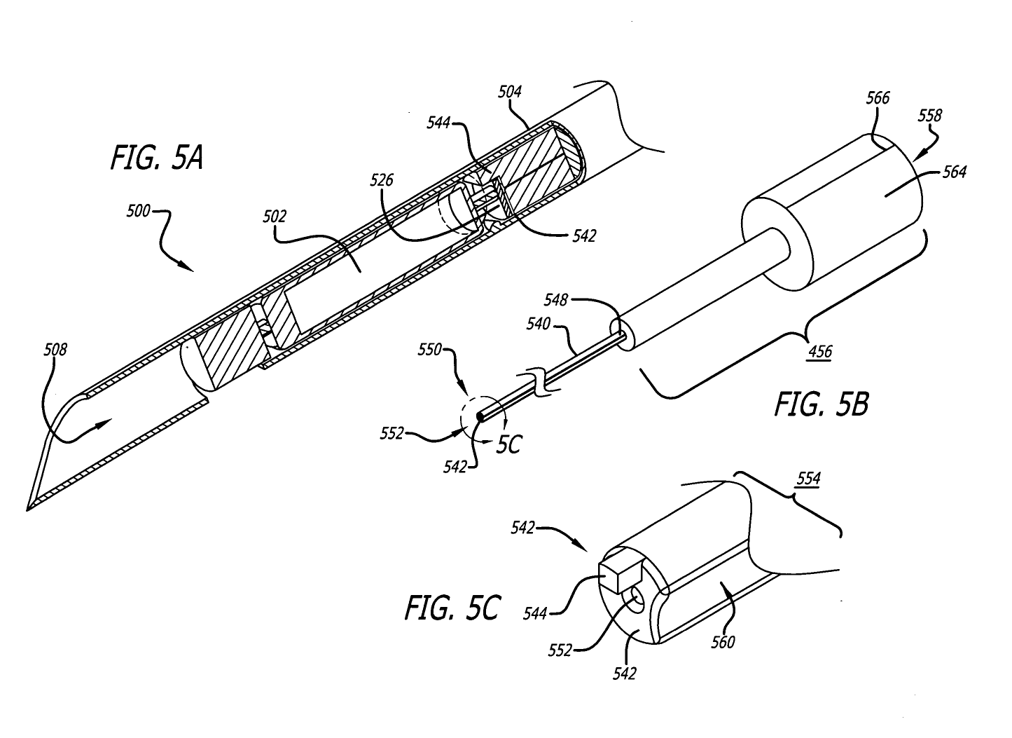

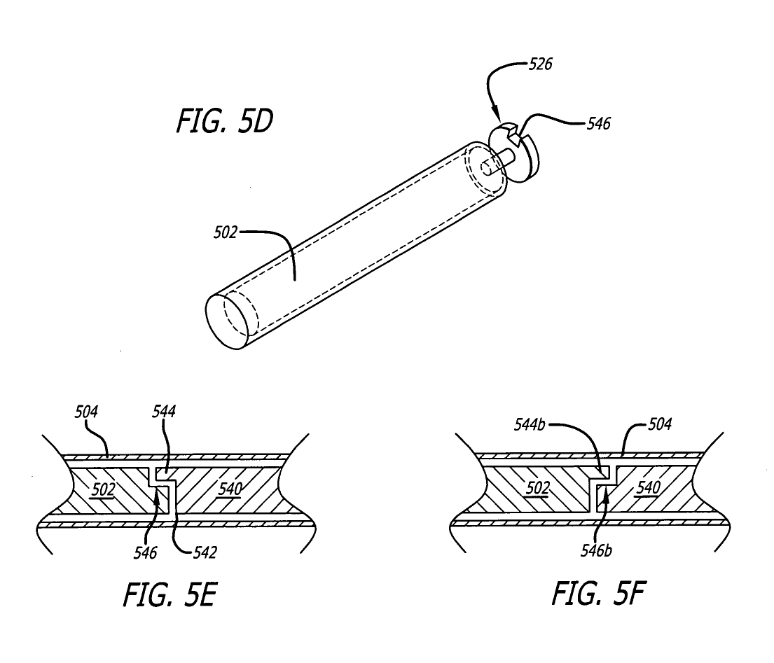

As depicted in FIG. 5A, in one embodiment the injection device (500) may include a cannula (504), a implant (502) positioned in the cannula lumen (508), and a probe (540) positioned such that an implant end surface abuts the probe distal end surface (542). Both the implant end surface (526) and probe distal end surface (542) may be configured to prevent the implant (502) from rotating with respect to the probe (540) while the surfaces abut. FIG. 5B depicts one embodiment in which the probe distal end surface is configured as a tab (544) having a cross-sectional shape, such as a rectangular tab (544) (FIG. 5C). The tab (544) may be formed integrally in the probe (540) or may be formed as a separate structure which is attached to the probe distal end surface (542). Further, the implant end surface (526) may be configured as a slot (546) having a cross-sectional shape selected to be compatible with the tab cross-sectional shape, such as a rectangular slot (546) (FIGS. 5D & E).

In one embodiment, the invention may include a method for positioning an implant such as a implant or sensor proximate to a testing position including, a proximal urethral afferent, dorsal penile nerve or clitoral nerve, at which the implant will function effectively including: (a) inserting a cannula distal tip having a implant retained in the cannula lumen into the body until the implant reaches a testing position; (b) testing the implant while within the cannula lumen at the testing position to determine whether the implant is functioning effectively; (c) discharging the implant from the lumen of the cannula at the testing location if the testing reveals that the implant is functioning effectively at the test location. This method may be utilized to pre-test the implant itself at the testing position prior to releasing it from the injection device.

In one embodiment, the method may further include moving the cannula containing the implant to a new test location, if testing shows that the implant is not located at an effective position, and re-testing the implant while within the cannula lumen at the new testing position to determine whether the implant is functioning effectively. In some methods, movement of the implant to a new test location may comprise moving the implant longitudinally relative to the target location. In some methods, movement of the implant to a new test location may comprise rotating the implant axially relative to the target location.

In these embodiments testing of the implant may comprise any activity which is useful in assessing that the implant has been properly placed relative to the target tissue and/or that the implant is functioning effectively to achieve the desired result. In one embodiment, the implant is a implant and testing of the implant may include delivery of a signal(s) to the implant. In one example of this embodiment testing may consist of the delivery of a command signal to an implant from an external controller. Further, the command signal may be transmitted to the implant using electromagnetic radiation. Upon receipt of the command signal, the implant may generate an electrical stimulation current which is applied to the surrounding tissues via electrodes at the two ends of the implant. If the implant is correctly placed and functioning in or near a muscle or muscle nerve, the operator may observe the contraction thereby induced in the muscle, confirming the placement and function of the implant.

In one embodiment, the implant is discharged from the cannula lumen at the testing location by maintaining position of implant at testing location while cannula is withdrawn. Further, the longitudinal and/or axial position of the implant may be maintained relative to the testing location when the implant is discharged. For example, in discharging the implant a probe may be used to stabilize the implant while a cannula is withdrawn to expose the implant at the tested location.

For example, a microstimulator may be roughly positioned using an ultrasound probe in conjunction with a cystourethrogram, and finely positioned longitudinally and axially, and stimulation patterns tested using testing of the microstimulator in the target location to achieve the desired functional effect of micturition or continence before the microstimulator is released from the “BION Insertion Tool” (33).

In one embodiment, for a male patient, an injection device, such as a cannula or needle (33) may be passed through about the midline raphe of the perineum (35) (such as in between the scrotum and the anus). This implantation approach may enable access to the prostate (4) via a direct path while minimizing contact with other significant pelvic structures.

In one embodiment, for a female patient, a an injection device, such as a cannula or needle (33) may be used to access the proximal peri-urethra. In females however this may be via a peri-urethral vaginal approach rather than transperinealy, and may also include per vaginum examination as needed. However, a microstimulator positioned in or near the urethral sphincter may stimulate both the desired urethral afferents and the motor neurons that produce sphincter contraction. In one embodiment, low frequency stimulation (1-2 pps) may be used to elicit the spinal micturition reflex by the proximal urethral afferents. This may be advantageous in that low frequency stimulation may produce only brief, weak twitches of the external urethral sphincter (5) that would not interfere significantly with urine flow.

In one embodiment of this technique, a physician may utilize imaging techniques to facilitate accurate implantation of the microstimulator in males or females. For example, an ultrasound or other imaging probe (39) may be used transvaginally (in the female) or transrectally (in males or females) to visualize the anatomical structures of the pelvic area during implantation. In another example, a urethral catheter (31) may be placed to aid localization and/or facilitate the infusion of contrast dye or fluid into the urethra and or bladder to act as a guide or landmark for peri-urethral localization.

Sensing. In one embodiment, the device may also include the use of an implantable sensor (10) (including wireless, implantable sensors such as sensory BIONs) to detect bladder pressure, volume, distension or other indicators of bladder emptiness/fullness in order to alert the user to the need to empty the bladder. In one embodiment, the sensor may send control signals directly to a microstimulator (9 or 17) to induce micturition or continence, respectively.

In one embodiment, implants may be used in pairs to measure the physical distension of the bladder. In one embodiment, a microstimulator (9) may be used in conjunction with a sensor (10). The implantable sensor (10) may be attached near the dome of bladder (1), in or on the bladder wall, where the distance from implantable sensor (10) to microstimulator (9) may increase as the bladder becomes distended by urine. Such a placement and attachment can be achieved transperitoneally via laparoscopic surgery (not illustrated) or other methods known to those skilled in the medical arts.

In one embodiment, the distance between the two implants (9) and (10) is determined by having one implant emit a signal whose received strength can be detected by the other implant and varies as a monotonic function of the distance between them. Methods for the emission and detection of electrical and magnetic signals are taught in U.S. Pat. No. 6,658,297, which is incorporated herein by reference.

One such method may include having microstimulator (9) emit a pulse of current similar to that which is normally emitted to stimulate urethral afferents but with an amplitude, duration or frequency that does not effectively induce the micturition reflex. This electrical current will induce potential gradients throughout the surrounding electrically conductive tissues of the pelvis including the bladder. The local strength of said potential gradients decreases rapidly with increasing distance between the source of the electrical current (microstimulator 9) and the location of the sensor (10). The strength of the signal detected by the sensor may be transmitted to a controller 23 (which may be external to the patient or within microstimulator (9)) which may use the information about the distance and electrical coupling between the implants to infer the degree of bladder distention. This information may be conveyed to a patient through the user interface (25), so the user may decide to empty the bladder.

In one embodiment, the user interface (25) may receive and display information about the strength of the signal detected by sensor (10) so that a user may respond by effecting a command to facilitate the emptying of the bladder. Microstimulators incorporating sensing functions may be used for this purpose, as is described in U.S. patent application Ser. No. 10/200,273, filed Jul. 22, 2002 and U.S. Pat. No. 6,658,297, herein incorporated by reference. In one embodiment, the sensor (10) may directly signal a command to the microstimulators (9) or (17) to induce urination or prevent incontinence respectively, without the intervention of an external controller (23). Various combinations of signal emission and detection between two or more implanted devices such as those illustrated at locations (9), (10) and (17) may be utilized in a similar manner within the scope of this invention. Implantable sensor (10) may receive power and data via means applicable to microstimulators (9) and (17) as described above.

The descriptions of exemplary and anticipated embodiments of the invention have been presented for the purpose of illustration and description. They are not intended to be exhaustive or to limit the invention to the precise forms disclosed. Modifications and variations are possible in light of the teachings herein.

1. An apparatus for exciting proximal urethral afferents to induce micturition comprising:
a first injectable microstimulator comprising:
a chamber separating a plurality of exposed electrodes for delivering controllable electrical current in the area of the proximal urethral afferents;
an electronic circuit within said chamber in communication with said electrodes for generating the controllable electrical current in response to a control signal;
a receiving antenna within said chamber in communication with said electronic circuit for receiving a control signal;
a controller for generating the control signals;
a transmitting antenna in communication with said controller for transmitting a control signal to said receiving antenna, wherein the control signal includes instructions to generate an electrical pulse having a frequency in the range of 2 to 20 pulses per second until the bladder is emptied.

2. The apparatus for exciting proximal urethral afferents to induce micturition as in claim 1 wherein said injectable microstimulator is adapted for implantation proximate to or within the prostate of a male patient.

3. The apparatus for exciting proximal urethral afferents to induce micturition as in claim 1 wherein said injectable microstimulator is adapted for implantation proximate to the internal urethral sphincter of a female patient.

4. The apparatus for exciting proximal urethral afferents to induce micturition as in claim 1 further including a second injectable microstimulator that is injectable in an area in a human body which inhibits micturition.

5. The apparatus of claim 4, further including a transmitting antenna in communication with said controller for transmitting a control signal to said receiving antenna, wherein the control signal includes instructions to generate an electrical pulses having a frequency of about 20 pulses per second for about 5 seconds followed by a rest of about 5 seconds until bladder emptying is desired.

6. The apparatus for exciting proximal urethral afferents to induce micturition as in claim 4 wherein said second injectable microstimulator is adapted for stimulation of the dorsal penile nerve.

7. The apparatus for exciting proximal urethral afferents to induce micturition as in claim 4 wherein said second injectable microstimulator is adapted for stimulation of the clitoral nerve.

8. The apparatus for exciting proximal urethral afferents to induce micturition as in claim 1 further including a second injectable microstimulator wherein said first injectable microstimulator is adapted for implantation in the prostate and said second injectable microstimulator is adapted for implantation proximate to the dorsal penile nerve.

9. The apparatus for exciting proximal urethral afferents to induce micturition as in claim 1 further including a second injectable microstimulator wherein said first injectable microstimulator is adapted for implantation proximate to the internal urethral sphincter and said second injectable microstimulator is adapted for implantation proximate to the clitoral nerve.

10. The apparatus for exciting proximal urethral afferents to induce micturition as in claim 8 wherein said transmitting antenna is a coil of a circumference suitable for positioning around the base of the penis for controlling said second injectable microstimulator, or for relocation to the perineal area for controlling said first injectable microstimulator; or a coil incorporated into the seat of a wheelchair or toilet or other that is of a form suited to control either or both of said first and second microstimulators; or a coil incorporated into or worn as a waist belt or other that is of a form suited to control either or both of said first and second microstimulators.

11. The apparatus for exciting proximal urethral afferents to induce micturition as in claim 1 further comprising a user interface including a user-activated control switch for initiating stimulation of the proximal urethral afferents in order to induce or maintain micturition.

12. An apparatus for exciting proximal urethral afferents to induce micturition comprising an injectable microstimulator having a plurality of exposed electrodes for delivering controllable electrical current having a frequency in the range of 2 to 20 pulses per second until the bladder is emptied in the area of the proximal urethral afferents which cause the urethral afferents to induce micturition by inducing at least one of contraction of the detrusor, relaxation of the internal urethral sphincter or relaxation of the external urethral sphincter.

13. An apparatus for exciting proximal urethral afferents to induce micturition comprising:
a first microstimulator implanted in proximity to proximal urethral afferents having a chamber separating a plurality of exposed electrodes for delivering controllable electrical current in the area of the proximal urethral afferents in response to at least one control signal and a receiving antenna within said chamber for receiving at least one control signals;
a control unit for generating at least one control signal; and
a transmitting antenna in communication with said control unit for transmitting the at least one control signals to said receiving antenna, wherein the control signal includes instructions to generate an electrical pulse having a frequency from about 2 to 20 pulses per second until the bladder is emptied.

14. The apparatus for exciting proximal urethral afferents to induce micturition as in claim 13 wherein the first microstimulator is configured for implantation proximate to or within the prostate, and a second microstimulator is configured for implantation proximate to the dorsal penile nerve.

15. The apparatus for exciting proximal urethral afferents to induce micturition as in claim 12 wherein the first microstimulator is configured for implantation proximate to the internal urethral sphincter, and a second microstimulator is configured for implantation proximate to the clitoral nerve.

16. The apparatus for exciting proximal urethral afferents to induce micturition as in claim 11 wherein said transmitting antenna is a coil, wherein said coil is of a circumference suitable for positioning around the base of the penis and adapted for controlling said first microstimulator and a second microstimulator.

17. The apparatus for exciting urethral afferents to induce micturition as in claim 11 further comprising a user interface including a user-activated control switch for initiating stimulation of the proximal urethral afferents in order to induce or maintain micturition.

18. A method for inducing micturition comprising:
positioning a leadless microstimulator proximate the proximal urethra; and
electrically stimulating a proximal urethral afferent to induce at least one of contraction of the detrusor, relaxation of the internal urethral sphincter or relaxation of the external urethral sphincter.

19. A method for exciting proximal urethral afferents to induce micturition comprising:
generating an electrical signal by a control unit;
delivering the signal to a transmitting antenna;
receiving the signal by a receiving antenna within a first microstimulator in the area of urethral proximal afferents;
generating an electrical signal in the first microstimulator in the area of urethral afferents having a frequency in the range of 2 to 20 pulses per second until the bladder is emptied and
inducing micturition.

20. The method for exciting proximal urethral afferents to induce micturition as in claim 19 further comprising delivering an electrical signal to a proximal urethral afferent through a plurality of microstimulators.

21. The method for exciting proximal urethral afferents to induce micturition as in claim 19 further comprising inhibiting micturition with a second microstimulator that receives a signal from the control unit and generates an electrical signal for inhibiting micturition.

22. The method for exciting urethral afferents to induce micturition as in claim 19 further comprising initiating stimulation of the urethral afferents in order to induce or maintain micturition.

23. The method for exciting proximate urethral afferents to induce micturition as in claim 19 wherein the first microstimulator is implanted in the prostate in a male patient.

24. The method for exciting proximate urethral afferents to induce micturition as in claim 17 wherein the first microstimulator is implanted proximate to internal urethral sphincter in a female patient.

25. A method for creating an electronic interface to proximate urethral afferents to induce micturition comprising injecting a first microstimulator in the area of the proximal urethral afferents.

26. The method for creating an electronic interface to urethral afferents to control micturition as in claim 25 further comprising injecting a second microstimulator in the area of the dorsal penile nerve to inhibit micturition.

27. The method for creating an electronic interface to urethral afferents to control micturition as in claim 25 further comprising injecting a second microstimulator into a human recipient in the area of the clitoral nerve to inhibit micturition.

28. The method for creating an electronic interface to urethral afferents to control micturition as in claim 25 wherein said injecting is through an injection device.

29. A method of implanting a microstimulator proximate to a proximal urethral afferent nerve in a male patient comprising:
inserting an injection device having a cannula through the perineum;
positioning the microstimulator in the injection device cannula proximate to an urethral afferent in the prostate; and
releasing the microstimulator from the injection device cannula proximate to the urethral afferent in the prostate.

30. The method of claim 29 further comprising prior to releasing the microstimulator from the injection device cannula proximate to the urethral afferent in the prostate, testing the microstimulator for excitation of the urethral afferents by observing at least one of the contraction of the detrusor or relaxation of the urethral sphincter.

31. A method of implanting a microstimulator proximate to a proximal urethral afferent nerve in a female patient comprising:
inserting an injection device having a cannula through the vagina;
positioning the microstimulator in the injection device cannula proximate to an urethral afferent proximate to the internal urethral sphincter; and
releasing the microstimulator from the injection device cannula proximate to the urethral afferent proximate to the internal urethral sphincter.

32. The method of claim 31 further comprising prior to releasing the microstimulator from the injection device cannula proximate to the urethral afferent proximal to the internal urethral sphincter, testing the microstimulator for excitation of the urethral afferents by observing at least one of the contraction of the detrusor or relaxation of the internal or external urethral sphincter.

33. A method for positioning a microstimulator proximate to the proximal urethral afferents to induce micturition comprising:
inserting a distal tip of a cannula having the microstimulator retained within a cannula lumen through the perinueum until the implant reaches a testing position proximate to the proximal urethral afferents;
testing the microstimulator while within the cannula lumen at the testing position to determine whether the implant is functioning effectively to induce micturition; and
discharging the microstimulator from the lumen of the cannula at the testing location if the testing reveals that the implant is functioning effectively at to induce micturition.

34. The method of claim 33, further including moving the microstimulator within the cannula lumen to a new position if testing shows the implant is not located at effective position and re-testing the implant while within the cannula lumen at the testing position to determine whether the implant is functioning effectively to induce micturition.

35. The method of claim 34 wherein the movement of the microstimulator includes longitudinal movement relative to the target location.

36. The method of claim 34 wherein the movement of the microstimulator includes axial rotation relative to the target location.

37. The method of claim 34, wherein the microstimulator is maintained at the testing location during the discharge of the implant from the lumen.

38. A method for measuring bladder distention and controlling micturition comprising:
implanting a first microstimulator proximate to the bladder wall;
implanting a second microstimulator proximate to a proximal urethral afferent;
emitting a electrical pulse from one of the first or second microstimulator;
detecting the strength of the electrical field at the other of the first or second microstimulator;
calculating the distension of the bladder from the strength of the electrical field detected to determine the presence of urine in the bladder; and
emitting an electrical pulse from the second microstimulator to excite the proximal urethral afferent to induce micturition if urine is present in the bladder.

39. The method of claim 38 wherein the first microstimulator is implanted proximate to the dome of the bladder.

40. The method of claim 38 wherein information reflecting the strength of the electrical field detected is conveyed to an external controller for calculating the distention of the bladder.

41. The method of claim 38 wherein a result of the calculation of the distention of the bladder is transmitted to a user interface.

42. The method of claim 38 further comprising emitting an electrical pulse at a third microstimulator implanted proximate to a dorsal penile nerve to inhibit micturition.

43. The method of claim 38 further comprising emitting an electrical pulse at a third microstimulator implanted proximate to a clitoral nerve to inhibit micturition.
